(19) United States
(12) Patent Application Publication
(10) Pub. No.: US 2026/0053557 A1
(43) Pub. Date: Feb. 26, 2026

(54) INTEGRATED ASSEMBLY HAVING AN ELECTROCAUTERY DEVICE

(71) Applicant: Boston Scientific Scimed, Inc., Maple Grove, MN (US)

(72) Inventors: Lauren KOON, Mississauga (CA); Christian BALKOVEC, Cambridge (CA)

(21) Appl. No.: 19/296,361

(22) Filed: Aug. 11, 2025

Related U.S. Application Data
(60) Provisional application No. 63/683,109, filed on Aug. 14, 2024.

(51) Int. Cl. A61B 18/14; A61B 18/00

(52) U.S. Cl. CPC A61B 18/1492; A61B 2018/00077; A61B 2018/00083; A61B 2018/00178; A61B 2018/00184; A61B 2018/00351; A61B 2018/00708; A61B 2018/00922; A61B 2018/0094; A61B 2018/1425

(57) ABSTRACT

A system for perforating a tissue within a heart is disclosed. The system includes a delivery device comprising an elongated body having a body length, a proximal end and an opposite distal end portion terminating in a distal tip, a handle connected to the proximal end of the elongated body, and a lumen extending through the handle and the elongated body and dimensioned to slidingly receive a perforation device. The system further includes an electrocautery device removably positioned into a void on the handle and connected to an energy source. The electrocautery device also includes a conductive member positioned inside the handle and in physical communication with the perforation device positioned within the lumen. When the electrocautery device is activated, energy is delivered from the electrocautery device through the conductive member and to the distal tip of the perforation device.

### CROSS REFERENCE TO RELATED APPLICATIONS

The present application claims priority to U.S. Provisional Patent Application No. 63/683,109 filed Aug. 14, 2024, the disclosure of which is incorporated herein in its entirety.

### TECHNICAL FIELD

The present disclosure relates to medical systems and methods for accessing a tissue within a heart of a patient. More specifically, the present disclosure relates to an integrated assembly having a puncture device and an electrocautery device configured to facilitate radiofrequency puncture of a tissue within a heart of a patient.

### BACKGROUND

Current commercially available devices for integrated needle-based transseptal puncture and dilation are cumbersome and difficult to control for an operator. For example, current dilators with integrated needles do not enable the operator to maintain a desired position during transseptal puncture while also activating the application of electrocautery. An operator must simultaneously maintain the position on the septum, maintain dilator and sheath alignment, apply and maintain pressure to advance the needle, apply forward pressure to puncture and dilate the septum, while also optionally apply electrocautery-which generally requires a second person to activate a switch. In some circumstances, the mechanism to activate electrocautery is via an electrosurgery pencil or a foot pedal. The use of a foot pedal may not be desirable as it can be activated accidentally, in some circumstances. In other instances, the electrosurgery pencil cannot easily be activated while holding and maintaining position of the transseptal assembly. In these circumstances, operators have been known to cross their hands while applying the current configurations.

Risks associated with the current design include procedure complications like a suboptimal transseptal puncture location which increases procedure complexity. Another risk associated with the current design is tamponade. Cardiac tamponade refers to a medical emergency in which fluid accumulates in the pericardium, i.e., the sac surrounding the heart. This accumulation of fluid leads to increased pressure on the heart, which restricts its ability to pump blood effectively.

Against this background, there exists a continuing need in the industry to provide improved devices and methods for gaining access to and perforating a tissue within a patient's heart. An object of the present invention is therefore to provide such an apparatus.

### SUMMARY

In Example 1, a system for perforating a tissue within a heart includes a delivery device comprising an elongated body having a body length, a proximal end and an opposite distal end portion terminating in a distal tip, a handle having a handle length and connected to the proximal end of the elongated body, and a lumen extending through the handle and the elongated body and dimensioned to slidingly receive a perforation device having a perforation device body, a perforation device proximal end and an opposite perforation device distal end portion terminating in a distal tip, wherein the delivery device body length and the handle length together define a delivery device length. The system also includes an electrocautery device removably positioned into a void on the handle and connected to an energy source, the electrocautery device having a conductive member positioned inside the handle and in physical communication with the perforation device positioned within the lumen; wherein when the electrocautery device is activated, energy is delivered from the electrocautery device through the conductive member and to the distal tip of the perforation device.

Example 2 is the system of Example 1 wherein the electrocautery device is an accessory device.

Example 3 is the system of Example 1 wherein the perforation device includes at least one or more grooves located on the perforation device body; and the conductive member aligns with one of the at least one or more grooves within the perforation device body to create a physical communication with the perforation device.

Example 4 is the system of Example 1 wherein when the electrocautery device is activated the physical communication between the conductive member and the perforation device allows for electrical communication with the distal end of the perforation device to facilitate radiofrequency puncture of a tissue within the heart.

Example 5 is the system of Example 1 further comprising a needle advancement mechanism positioned on the delivery device.

Example 6 is the system of Example 5 wherein the needle advancement mechanism is configured to interact with the perforation device and advance the perforation device longitudinally within the delivery device lumen.

Example 7 is the system of Example 6 wherein the perforation device includes at least one or more grooves located on the perforation device body; the needle advancement mechanism is a knob having a first portion protruding from the delivery device and a second portion positioned within the lumen; and when the knob aligns with one of the at least one or more grooves within the perforation device body, a user longitudinally advances the first portion of the knob across the delivery device thereby advancing the perforation device longitudinally within the lumen.

Example 8 is the system of Example 6 wherein the needle advancement mechanism is a dial having a traction element, and wherein friction applied by the dial to the perforation device advances the perforation device longitudinally within the lumen.

Example 9 is the system of Example 1 wherein the electrocautery device includes a first button, and wherein the electrocautery device is activated when a user presses the first button.

Example 10 is the system of Example 1 wherein the electrocautery device includes a second button, and wherein when the second button is pressed by a user the electrocautery device stops delivery of energy to the conductive member.

Example 11 is the system of Example 1 wherein the perforation device is insulated along a portion of the perforation device body.

Example 12 is the system of Example 1 wherein the electrocautery device is held in place within the void of the handle by friction.

Example 13 is the system of Example 1 wherein the electrocautery device is held in place within the void of the handle by at least one or more actuatable elements which prevent unintended dislodgement of the electrocautery device.

Example 14 is the system of Example 13 wherein the at least one or more actuatable elements include latches, switches and screw features.

Example 15 is the system of Example 1 wherein the delivery device is a dilator.

In Example 16, a system for perforating a tissue within a heart includes a delivery device comprising an elongated body having a body length, a proximal end and an opposite distal end portion terminating in a distal tip, a handle having a handle length and connected to the proximal end of the elongated body, and a lumen extending through the handle and the elongated body and dimensioned to slidingly receive a perforation device having a perforation device body, a perforation device proximal end and an opposite perforation device distal end portion terminating in a distal tip, wherein the delivery device body length and the handle length together define a delivery device length. The system also includes an electrocautery device removably positioned into a void on the handle and connected to an energy source, the electrocautery device having a conductive member positioned inside the handle and in physical communication with the perforation device positioned within the lumen; wherein when the electrocautery device is activated, energy is delivered from the electrocautery device through the conductive member and to the distal tip of the perforation device.

Example 17 is the system of Example 16 wherein the electrocautery device is an accessory device.

Example 18 is the system of Example 16 wherein the delivery device is a dilator.

Example 19 is the system of Example 16 wherein the perforation device includes at least one or more grooves located on the perforation device body; and the conductive member aligns with one of the at least one or more grooves within the perforation device body to create a physical communication with the perforation device.

Example 20 is the system of Example 16 wherein when the electrocautery device is activated the physical communication between the conductive member and the perforation device allows for electrical communication with the distal end of the perforation device to facilitate radiofrequency puncture of a tissue within the heart.

Example 21 is the system of Example 16 further comprising a needle advancement mechanism positioned on the delivery device, and wherein the needle advancement mechanism is configured to interact with the perforation device and advance the perforation device longitudinally within the lumen.

Example 22 is the system of Example 16 wherein the electrocautery device includes a first button, and wherein the electrocautery device is activated when a user presses the first button.

Example 23 is the system of Example 16 wherein the electrocautery device includes a second button, and wherein when the second button is pressed by a user the electrocautery device stops delivery of energy to the conductive member.

Example 24 is the system of Example 16 wherein the perforation device is insulated along a portion of the perforation device body.

Example 25 is the system of Example 16 wherein the electrocautery device is held in place within the void of the handle by friction.

In Example 26, a device for perforating a tissue within a heart includes a dilator comprising an elongated body having a body length, a proximal end and an opposite distal end portion terminating in a distal tip, a handle having a handle length and connected to the proximal end of the elongated body, and a dilator lumen extending through the handle and the elongated body and dimensioned to slidingly receive a perforation needle having a perforation needle body, a perforation needle proximal end and an opposite perforation needle distal end portion terminating in a distal tip, wherein the dilatory body length and the handle length together define a dilator length. The device also includes an electrocautery device removably positioned into a void on the dilator handle and connected to an energy source, the electrocautery device having a conductive member positioned inside the dilator handle and in physical communication with the perforation needle positioned within the dilator lumen. The device further includes a needle advancement mechanism positioned on the dilator and in physical communication with the perforation device, the needle advancement mechanism configured to advance the perforation needle longitudinally within the dilator lumen; wherein when the electrocautery device is activated, energy is delivered from the electrocautery device through the conductive member and to the distal tip of the perforation device.

Example 27 is the device of Example 26 wherein the electrocautery device is an accessory device.

Example 28 is the device of Example 26 wherein the needle includes at least one or more grooves located on the needle body; and the conductive member aligns with one of the at least one or more grooves within the needle body to create a physical communication with the perforation device.

Example 29 is the device of Example 26 wherein the needle includes at least one or more grooves located on the needle body; the needle advancement mechanism is a knob having a first portion protruding from the dilator and a second portion positioned within the dilator lumen; and when the knob aligns with one of the at least one or more grooves within the needle body, a user longitudinally advances the first portion of the knob across the dilator thereby advancing the needle longitudinally within the dilator lumen.

Example 30 is the device of Example 26 wherein the needle advancement mechanism is a dial having a traction element, and wherein friction applied by the dial to the needle advances the needle longitudinally within the dilator lumen.

Example 31 is the device of Example 26 wherein the needle advancement mechanism is an accessory device.

Example 32 is the device of Example 26 wherein the needle is insulated along a portion of the needle body.

Example 33 is the device of Example 26 wherein the electrocautery device is held in place within the void of the dilator handle by at least one or more actuatable elements which prevent unintended dislodgement of the electrocautery device.

Example 34 is the device of Example 33 wherein the at least one or more actuatable elements include latches, switches and screw features.

In Example 35, a method for perforating a tissue within a heart includes providing a delivery device comprising an elongated body having a body length, a proximal end and an opposite distal end portion terminating in a distal tip, a handle having a handle length and connected to the proximal end of the elongated body, and a lumen extending through the handle and the elongated body and dimensioned to slidingly receive a perforation device having a perforation device body, a perforation device proximal end and an opposite perforation device distal end portion terminating in a perforation device distal tip, wherein the delivery device body length and the handle length together define a delivery device length. The method for perforating a tissue within a heart also includes providing an electrocautery device removably positioned into a void on the handle and connected to an energy source, the electrocautery device having a conductive member positioned inside the handle and in physical communication with the perforation device positioned within the lumen; wherein when the electrocautery device is activated, energy is delivered from the electrocautery device through the conductive member and to the distal tip of the perforation device.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, in accordance with embodiments of the present disclosure.

FIG. 2 is a schematic illustration of a system for perforating a tissue within a heart having a delivery device comprising a void on a handle of the device, the delivery device dimensioned to slidingly receive a perforation device, in accordance with embodiments of the present disclosure.

FIGS. 3A-3B are schematic illustrations of an electrocautery device positioned within the void of the delivery device of FIG. 2, in accordance with embodiments of the present disclosure.

FIGS. 4A-4C are schematic illustrations of the delivery device of FIGS. 2-3B in which a conductive member of the electrocautery device is in physical communication with the perforation device of the delivery device, in accordance with embodiments of the present disclosure.

FIGS. 5A-5C are schematic illustrations of a needle advancement mechanism configured to interact with the system of FIGS. 2-4C, in accordance with embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

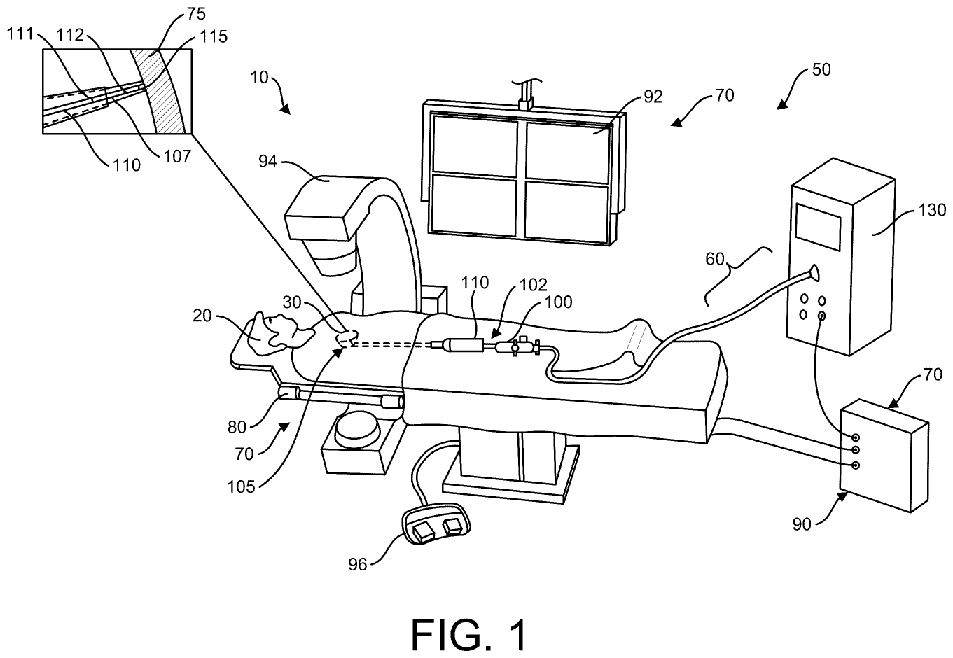

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The electroporation catheter system 60 includes a catheter 100 having a proximal portion 102 and a distal portion 105, an introducer sheath 110, and an electroporation console 130. Additionally, the electroporation catheter system 60 includes various connecting elements, e.g., cables, umbilical, and the like, that operate to functionally connect the components of the electroporation catheter system 60 to one another and to the components of the EAM system 70. This arrangement of connecting elements is not of critical importance to the present disclosure, and the skilled artisan will recognize that the various components described herein can be interconnected in a variety of ways.

In some embodiments, the introducer sheath 110 is operable to provide a delivery conduit through which the electroporation catheter 100, in particular all or part of the distal portion 105 thereof, can be deployed to the specific target sites within the patient's heart 30.

In some embodiments, the electroporation catheter system 60 is configured to deliver electric field energy to targeted tissue in the patient's heart 30 to create tissue apoptosis, rendering the tissue incapable of conducting electrical signals.

The electroporation console 130 is configured to control functional aspects of the electroporation catheter system 60. In embodiments, the electroporation console 130 includes one or more controllers, microprocessors, and/or computers that execute code out of memory to control and/or perform the functional aspects of the electroporation catheter system 60. In embodiments, the memory can be part of the one or more controllers, microprocessors, and/or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the electroporation catheter 100 to generate electric fields sufficient to achieve the desired clinical effect, in particular ablation of target tissue through irreversible electroporation. In embodiments, the electroporation console 130 can deliver the pulsed waveforms to the catheter 100 in a monopolar or bipolar mode of operation, as will be described in further detail herein.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 can be the OPAL™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In some embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned OPAL HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

In an embodiment, the exemplary clinical setting 10 may be used for a medical procedure within a patient's heart for gaining transseptal access, as well as access to the epicardial space. In an example, procedures for providing access to the left atrium use transseptal access systems and devices for subsequent deployment of secondary diagnostic and/or therapeutic devices within the left atrium. In these procedures, a target tissue site can be defined by tissue on the atrial septum 75. The target site is accessed via the inferior vena cava (IVC), for example through the femoral vein, according to conventional catheterization techniques. In other embodiments, access to the target site on the atrial septum 75 may be accomplished using a superior approach wherein the transseptal access system is advanced into the right atrium via the superior vena cava (SVC). As is known, the human heart 30 has four chambers, a right atrium, a left atrium, a right ventricle and a left ventricle. Separating the right atrium and the left atrium is an atrial septum 75 and separating the right ventricle and the left ventricle is a ventricular septum. As is further known, deoxygenated blood from the patient's body is returned to the right atrium via an inferior vena cava (IVC) or a superior vena cava (SVC).

In some embodiments, transseptal access procedures may include many devices like an introducer sheath 110, a dilator 107, a puncture device 111 having a distal end portion 112 terminating in a tip electrode 115, and a guidewire (not shown). In various embodiments, the puncture device 111 may be a mechanical puncture device (e.g., a needle) or a radiofrequency (RF) perforation device. In other embodiments, as will be discussed in greater detail below, an electrocautery device may be connected to the puncture device 111 to facilitate in RF perforation. The puncture device 111 can be disposed within the dilator 107, which itself can be disposed within the sheath 110. In one embodiment in which the transseptal access system is deployed into the right atrium via the IVC, a user introduces a guidewire (not shown) into a femoral vein, typically the right femoral vein, and advances it towards the heart 30. The sheath 110 may then be introduced into the femoral vein over the guidewire, and advanced towards the heart 30. In one embodiment, the distal ends of the guidewire and sheath 110 are then positioned in the SVC. These steps may be performed with the aid of an imaging system, as discussed above. The dilator 107 may then be introduced into the sheath 110 and over the guidewire, and advanced through the sheath 110 into the SVC. Alternatively, the dilator 107 may be fully inserted into the sheath 110 prior to entering the body, and both may be advanced simultaneously towards the heart 30.

When the guidewire, sheath 110 and dilator 107 have been positioned in the SVC, the guidewire is removed from the body, and the sheath 110 and the dilator 107 are retracted so that their distal ends are positioned in the right atrium. In an embodiment, the puncture device 111 described can then be introduced into the dilator 107, and advanced toward the heart 30. In other embodiments, the puncture device 111 described may be introduced prior to the retraction of the sheath 110 and the dilator 107 from the SVC into the right atrium. The puncture device 111 is then positioned such that the tip electrode 115 is aligned with or protruding slightly from the distal end of the dilator 107. In embodiments where the puncture device 111 is an RF perforation device, with the tip electrode 115 and dilator 107 positioned at the target site, energy is delivered from an energy source, e.g., an RF generator, through the RF perforation device 111 to the tip electrode 115 and the target site. In some embodiments, the energy is delivered at a power of at least about 5 W at a voltage of at least 200 V RMS, or in certain embodiments about 565 V (peak-to-peak), and functions to vaporize cells in the vicinity of the tip electrode 115, thereby creating a void or perforation through the tissue at the target site. The user then applies force to the RF perforation device 111 so as to advance the tip electrode 115 at least partially through the perforation. In these embodiments, when the tip electrode 115 has passed through the target tissue, that is, when it has reached the left atrium, energy delivery is stopped. In some embodiments, the step of delivering energy occurs over a period of between about 0.1 second and about 5 seconds. In other embodiments, the step of delivering energy occurs over a period of about 300 milliseconds.

The present disclosure describes novel systems and methods for providing safe access to the heart. As will be explained in greater detail herein, the embodiments of the present disclosure improve the functionality and effectiveness of integrated perforation device-based systems using an electrocautery device to puncture a tissue within a heart of a patient.

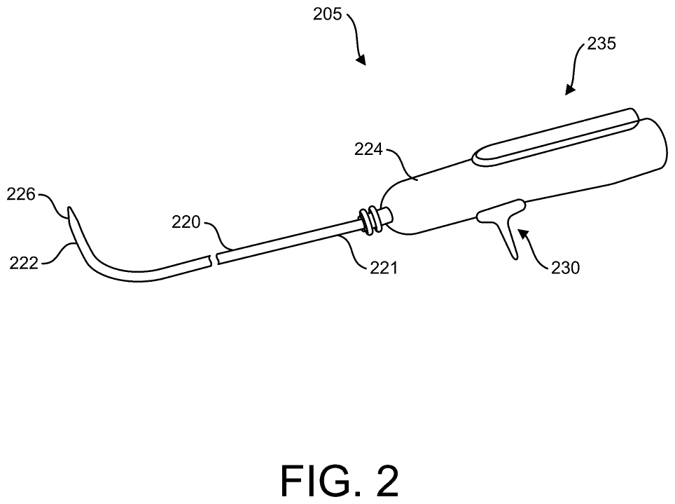

FIG. 2 is an illustration of an access system for perforating a tissue within a heart of a patient having a delivery device 205 dimensioned to slidingly receive a perforation device, in accordance with embodiments of the present disclosure. As shown, the delivery device 205 includes an elongated body 220 having a body length, a proximal end portion 221 and an opposite distal end portion 222 terminating in a distal tip 226. As further shown, the delivery device 205 may further include an ergonomic handle 224 having a handle length and connected to the proximal end 221 of the elongated body 220. In an embodiment, the body length and the handle length of the delivery device 205 together define a delivery device length. As will be discussed in greater detail below, the delivery device 205 further defines a lumen extending through the handle 224 and the elongated body 220 of the delivery device 205 and is dimensioned to slidingly receive a perforation device. In an embodiment, the perforation device includes a perforation device body, a perforation device proximal end and an opposite perforation device distal end portion terminating in a perforation device distal tip. In an embodiment, the perforation device is inserted into the handle 224, extends through the delivery device 205 and may exit the distal tip 226. In some embodiments, the perforation device may be any type of crossing device known in the art. In some embodiments, the perforation device may be an RF crossing device. In other embodiments, the perforation device may be a perforation needle. In other embodiments, the perforation device may be a mechanical perforation needle.

In an embodiment, the handle 224 of the delivery device 205 may further include a void 235 (i.e., an opening, a slot, a mating feature, or an aperture). In an embodiment, the void 235 may be a space in which a secondary device may be placed to physically connect the secondary device into the handle 224 of the device 205 and therefore to the delivery device 205 itself. In an embodiment, the void 235 may be placed on the device body 220 or any other desired location on the device 205. In an embodiment, as will be discussed in greater detail below, the void 235 creates an aperture through the handle 224 of the device 205 and creates a pathway to the lumen of the device 205 so that one or more secondary devices may be in physical communication with the lumen. In an embodiment, as will be discussed, the one or more secondary devices, inserted through the void 235, may have access to and in physical communication with the perforation device that extends through the lumen of the device 205. In an embodiment, the one or more secondary devices may include an electrocautery device.

In some embodiments, the delivery device 205 may be any device that may slidingly receive and allow for longitudinal translation of the perforation device. In an embodiment, the delivery device may be a dilator, a catheter, an introducer sheath, a guidewire, or any other medical delivery devices defining at least one or more hollow lumens within which the perforation device may be inserted. In an embodiment, as will be discussed in greater detail below, the delivery device 205 is a dilator. In an embodiment, as will be discussed in greater detail below, the delivery device 205 further includes a needle advancement mechanism 230.

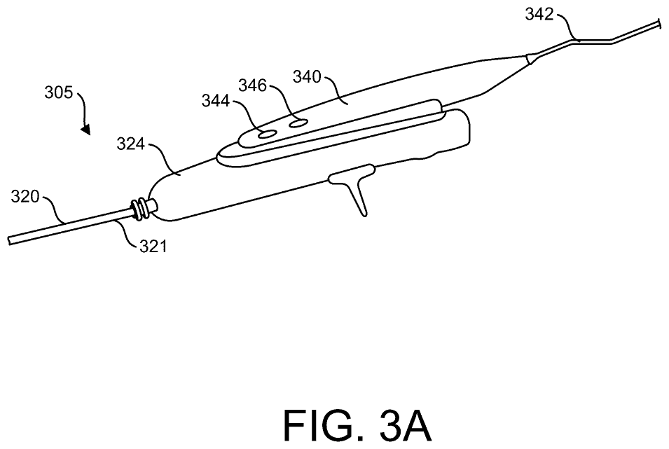

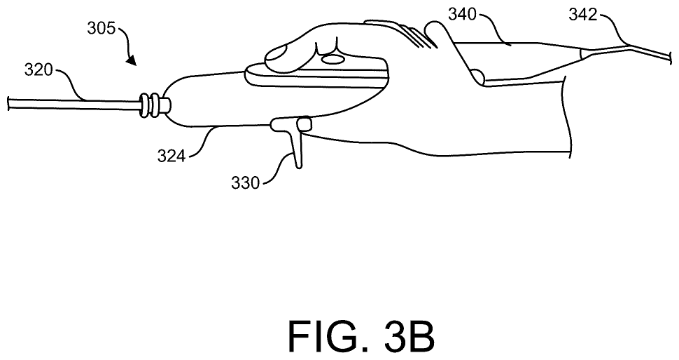

FIGS. 3A-3B are schematic illustrations of an electrocautery device 340 positioned into the void of the delivery device 305, in accordance with embodiments of the present disclosure. In an embodiment, the delivery device 305 may be substantially structurally and functionally identical to the delivery device 205 of FIG. 2, except as described in connection with FIGS. 3A-3B. In an embodiment, as discussed, the delivery device 305 includes an elongated body 320 and a handle 324 connected to the proximal end 321 of the elongated body 320. In an embodiment, as discussed, the handle 324 of the delivery device 305 may further include a void (i.e., an opening, slot, mating feature or aperture). As shown, the electrocautery device 340 is removably positioned into the void of the handle 324 of the device 305. In other embodiments, the electrocautery device 340 may be installed into the handle 324 and not be able to be removed by a user. In an embodiment, the shape of the void may be similar to the shape of the electrocautery device 340 so that the electrocautery device may be fitted into the void and therefore into the delivery device handle 324, becoming one ergonomic body which can be easily manipulated by a user. In an embodiment, as shown and discussed below, the electrocautery device 340 may further include at least one or more buttons, dials, or switches 344, 346 for electrocautery activation, stoppage, mode selection, variable power levels, among other needs.

In an embodiment, the electrocautery device 340 may be connected to an energy source, e.g., an RF generator which produces high-frequency electrical currents. In some embodiments, the electrocautery device 340 is connected to the energy source by a connecting wire 342 that supports the transfer of RF energy. In an embodiment, the electrocautery device 340 may be used to transfer radiofrequency energy from the energy source, through the perforation device and the perforation device distal tip and thus to a target site to perforate a target tissue.

In some embodiments, the perforation device may be any type of crossing device known in the art. In certain embodiments, the perforation device may be an RF crossing device. In other embodiments, the perforation device may be a perforation needle. In other embodiments, the perforation device may be a mechanical perforation needle. In an embodiment, the perforation device may not include a lumen or an open face so that coring is prevented when puncturing the target site.

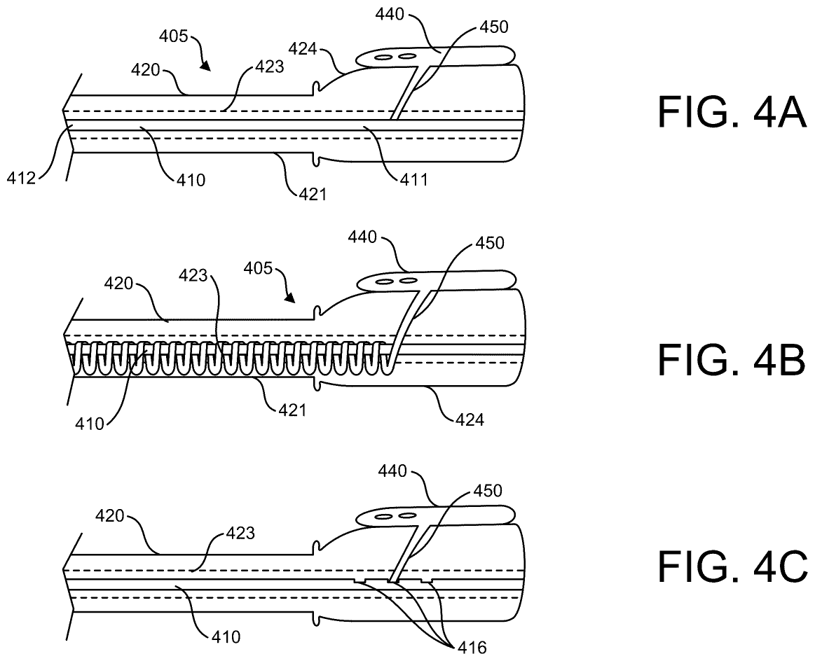

FIGS. 4A-4C are illustrations of the delivery device of FIGS. 2-3B in which a conductive member 450 of the electrocautery device 440 is in physical communication with a perforation device 410 of the delivery device 405, in accordance with embodiments of the present disclosure. In an embodiment, the delivery device 405 and electrocautery device 440 may be substantially structurally and functionally identical to the delivery device 205 and 305 and the electrocautery device 340 of FIGS. 2-3B, except as described in connection with FIGS. 4A-4C. In an embodiment, as discussed above, the delivery device 405 may include an elongate body 420 having a proximal end 421 and an opposite distal end portion. In an embodiment, the delivery device 405 further includes a lumen 423, as discussed above, extending through the handle 424 and the elongated body 420 of the delivery device 405 and dimensioned to slidingly receive a perforation device 410 having a perforation device body, a perforation device proximal end 411 portion and an opposite perforation device distal end 412 portion terminating in a perforation device distal tip.

In some embodiments, the perforation device 410 may be any type of crossing device known in the art. In certain embodiments, the perforation device may be an RF crossing device. In other embodiments, the perforation device may be a perforation needle. In other embodiments, the perforation device may be a mechanical perforation needle. In an embodiment, the perforation device may not include a lumen or an open face so that coring is prevented when puncturing the target site. In some embodiments, the delivery device 405 may include a primary lumen 423 and a secondary lumen. In some embodiments, the secondary lumen may be used for insertion of accessory devices, providing contrast into the medium, allow for media to be communicated with, among other needs. In an embodiment, the delivery device 405 may be dimensioned to slidingly receive the perforation device 410 through the secondary lumen instead of the primary lumen.

In an embodiment, as discussed above, the electrocautery device 440 may be connected to an energy source on one end through a wire. In an embodiment, as shown in FIGS. 4A-4C, the electrocautery device 440 may also include a conductive member 450. In an embodiment, once the electrocautery device 440 is inserted into the void of the handle of the delivery device, the conductive member 450 may be positioned inside the void of the handle 424 of the delivery device 405 and may access the lumen 423 of the delivery device 405. In an embodiment, the void is extended to the lumen 423 so that the conductive member 450 may be placed into the lumen 450 with ease. In an embodiment, once the perforation device 410 is inserted into the lumen 423 of the delivery device 405, the conductive member 450 may be in physical communication with the perforation device 410 positioned within the lumen 423. Thus, in certain embodiments, when the electrocautery device 440 is activated, the electrocautery device 440 is able to transfer radiofrequency energy from an external RF generator through the conductive member 450, to the perforation device 420 and to the distal tip of the perforation device 410 for subsequent delivery to a target tissue in a transseptal crossing or an epicardial and/or endocardial ablation procedure.

In certain embodiments, the electrocautery device 440 may be an accessory device. In other embodiments, the electrocautery device 440 may be pre-installed into the delivery device 405, and therefore not a separate component from the delivery device 405. In certain embodiments, when the electrocautery device 440 is activated, the physical contact between the conductive member 450 and the perforation device 410 allows for electrical communication with the perforation device 410, and therefore the distal end 412 of the perforation device 410, to facilitate radiofrequency puncture of a tissue within the heart. In an embodiment, the physical communication between the conductive member 450 and the perforation device 410 may include any form of connection that will allow electrical communication between the conductive member 450 and the perforation device 410 that is running through the lumen 423 of the delivery device 405. In an embodiment, the connection may include the conductive member 450 simply making any form of contact with the perforation device 410, as shown in FIG. 4A. In an embodiment, as shown in FIG. 4B, once the electrocautery device 440 is inserted into the void and therefore into the lumen 423, the conductive member 450 of the electrocautery device 440 may be able to wrap around the perforation device 410 to form a physical connection. In an embodiment, the number of windings (i.e., coils or loops) of the conductive member 450 around the perforation device 440 may vary depending on the procedure and as needed. In an embodiment, the conductive member 450 may wrap around the perforation device 410 only a few times, while in other embodiments, the conductive member 450 may wrap around the perforation device 410 many times. In an embodiment, the conductive member 450 may wrap around the perforation device 410 two to five times. In an embodiment, a conductive spring leaf or spring contacts, which in some embodiments are round to allow for translation, may be used to aid in the electrical-mechanical communication between the conductive member 450 and the perforation device 410. In yet another embodiment, as shown in FIG. 4C, the perforation device 410 may include at least one or more grooves 416 located on the perforation device body. In an embodiment, the conductive member 450 may align with one of the at least one or more grooves 16 within the perforation device body 410 to create a physical communication with the perforation device 410. In other embodiments, other methods of physical connection between the perforation device 410 and the conductive member 450 may be contemplated, including but not limited to, using a separate connector to connect the perforation device 410 to the conductive member 450, having a separate screw terminal, among others.

In an embodiment, the conductive member 450 may comprise any biocompatible electrically conductive material. Exemplary materials may include stainless steel, nickel-titanium alloy, and the like. Further, for ease of illustration, the distal portion 214 is depicted as a single solid structure, although the construction of the conductive member 450 can vary to accommodate the particular structural requirements for the delivery device 405 and the perforation device 410. For example, in some embodiments, the conductive member 450 can be constructed as a solid rod, a tube or a coil. In an embodiment, the perforation device 410 may be insulated along a portion of the perforation device body. In some embodiments, the proximal portion 411 of the perforation device 410, proximate the conductive member 450 and the perforation device 410 connection, may include an electrically insulated outer surface. As such, in certain embodiments, the proximal portion 411 can be handled directly by a user when the perforation device 410 is energized by the conductive member 450 and the electrocautery device 440. In some embodiments, the proximal portion 411 is of a unitary construction formed entirely of an electrically insulative material. In certain embodiments, one exemplary class of materials for construction of the insulated portion can include various grades of polytetrafluoroethylene (PTFE), polyetheretherketone (PEEK), Polyimide, among others. In some embodiments, the proximal portion 411 can further include reinforcing elements, e.g., a polymeric braid or coil, to enhance the structural properties, e.g., stiffness, torque transfer capability, and the like. In some embodiments, the proximal portion 411 is formed of a metal (e.g., a metal hypotube), and includes an outer electrically insulating layer.

In certain embodiments, as discussed above and as shown best in FIG. 3A, the electrocautery device 440 may include a first button 344. In an embodiment, the electrocautery device 440 may be activated, as thus is capable of transferring radiofrequency energy supplied by an external RF generator through the perforation device 410, when a user presses/actuates the first button 344. In some embodiments, the electrocautery device 440 may include a second button 346. In certain embodiments, when the second button 346 is pressed/actuated by a user the electrocautery device stops delivery of energy to the conductive member 450. In other embodiments, the electrocautery device 440 may include additional buttons used for other purposes, as discussed above. In some embodiment, one or more buttons may be used to increase or decrease the amount of energy transferred through the electrocautery device 340. In an embodiment, the buttons 344, 346 may be a dial, a switch or any other mechanism used for controlling transfer of energy. In some embodiments, the buttons 344, 346 allow for a single operator to maintain control of the assembly during all parts of the procedure without having to move hand positions excessively and be able to start, stop, or control the level of RF energy without having to move or adjust hand positions from the assembly.

In an embodiment, while the electrocautery device 440 is shown to be placed on the handle 424 of the delivery device 410, in other embodiments, the void and therefore the electrocautery device 440 may be placed in other locations on the delivery device 405. In an embodiment, the electrocautery device 440 is held in place within the void of the handle 424 by friction. In other embodiments, the electrocautery device may be held in place within the void of the handle 424 of the delivery device by at least one or more actuatable elements which prevent unintended dislodgement of the electrocautery device. In an embodiment, these elements create reversible mating features preventing translation. In an embodiment, the at least one or more actuatable elements can include latches, switches, screw features, among other elements in which allow for prevention of unintended dislodgement of the electrocautery device 440. In some embodiments, void and conductive mating features prevent translation and rotation of the electrocautery device 440, and reversible mating features secure the electrocautery device 440 from other degrees of freedom.

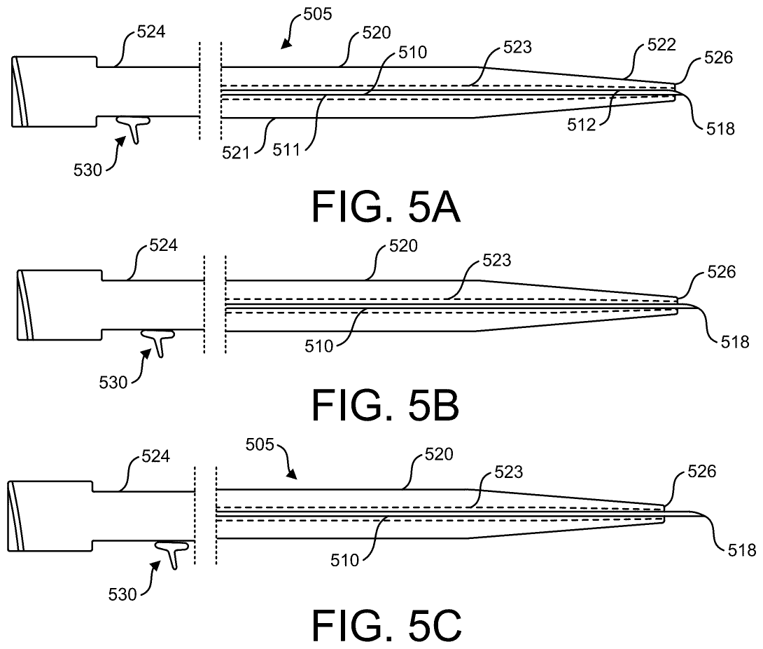

FIGS. 5A-5C are schematic illustrations of a delivery device 505 for puncturing a tissue within a heart having a needle advancement mechanism 530, in accordance with embodiments of the present disclosure. The device 505 may be substantially structurally and functionally identical to the device 205, 305, 405 of FIGS. 2-4C, except as described in connection with FIGS. 5A-5C. In an embodiment, the delivery device 505 may include a needle advancement mechanism 530 positioned on the delivery device 505. In an embodiment, as shown, the needle advancement mechanism 530 may be positioned on the handle 524 which enables extension of perforation device 510. In other embodiments, the needle advancement mechanism 530 may be positioned on the body 520 of the delivery device 505 as desired. In an embodiment, the needle advancement mechanism 530 is configured to interact with the perforation device 510 and advance the perforation device 510 longitudinally within the delivery device lumen 523. In some embodiments, as shown, the advancement mechanism 530 longitudinally selectively advances the perforation device 510 towards and past the distal portion 522 and the distal tip 526 of the delivery device body 520. In an embodiment, as shown, the delivery device 505 includes an elongate body 520 having a proximal end portion 521 and an opposite distal end portion 522 terminating in a distal tip 526. In an embodiment, the perforation device 510 includes a perforation device body, a perforation device proximal end portion 511 and a perforation device distal end portion 512 terminating in a distal tip 518. In an embodiment, the needle advancement mechanism 530 is an accessory device that may be attached to the delivery device 505 when desired to selectively extend the perforation device 510 from the distal tip 526 of the delivery device body 520. In an embodiment, the needle advancement mechanism 530 may be pre-installed to the device 505 so that it is not removable by the user.

In some embodiments, the advancement mechanism 530 may be a knob, a dial, a screw, a collar, or any other structure known in the art that may guide in advancing the perforation device 510. In some embodiments, the perforation device 510 may include at least one or more grooves located on the body of the perforation device 510. Additionally, in some embodiments, the advancement mechanism 530 may be a knob, a screw, or a switch having a top (or first) portion that protrudes from the handle 524 of the delivery device 505, so that a user may have access to the advancement mechanism 530, and a bottom (or second) portion positioned through the handle 524 and within the lumen 523. In certain embodiments, when the knob aligns with one of the at least one or more grooves within the perforation device 510, a user may longitudinally advance the first portion of the knob across the handle 524 thereby advancing the perforation device 510 longitudinally within the hollow lumen 523 of perforation device 510. Thus, in one embodiment, the perforation device 510 is selectively advanced by the advancement mechanism 530 on the handle 524.

In other embodiments, the advancement mechanism 530 may be a dial having a traction element, a top portion protruding from the handle 524 that is accessible to the user and a bottom portion positioned through the handle 524 and within the lumen 523 having access to perforation device 510. In certain embodiments, the friction applied by the dial to the perforation device 510 may advance the perforation device 510 longitudinally through the lumen 523, or a secondary lumen. Still other configurations known in the art may be contemplated to selectively advance the advancement mechanism 530 and thereby advancing the perforation device 510. In certain embodiments, the advancement mechanism 530 may be an accessory device that can be connected to the handle 524 to aid in advancing the perforation device 510.

In certain embodiments, the advancement mechanism 530 may further include an indicator mechanism (not shown) positioned on the handle 524. In other embodiments, the indicator mechanism may be positioned on the delivery device body (not shown). In an embodiment, the indicator mechanism indicates a protrusion length of the perforation device 510. Thus, in certain embodiments, the indicator mechanism may provide a user with feedback regarding the protrusion length of the perforation device 510 from the hollow lumen 523 of the delivery device 505. In some embodiments, the protrusion length is measured from the distal tip 526 of the device 505 to the distal tip 518 of the perforation device 510. In some embodiments, the indicator mechanism may indicate the length of the perforation device 510 from the distal tip of the device 505, while in other embodiments, the indicator mechanism may indicate meaningful protrusion points along the perforation device 510.

In certain embodiments, the indicator mechanism includes an indicator that may be a visual, a tactile or an auditory indicator. In certain embodiments, other indicators known in the art that provide communication to the user regarding the protrusion length of the perforation device 510 may be contemplated. By explicitly indicating the amount of protrusion of the perforation device 510 from the device 505, the user may more precisely tailor the protrusion length of the perforation device 510 to optimize the perforation device method that is being employed.

In one embodiment, the indicator mechanism may include an indicator light that communicates the protrusion length of the perforation device 510. In other embodiments, the indicator mechanism may include at least one or more tactile notches, or raised surface features, that allow a user to feel and/or observe the position of the perforation device 510. In still other embodiments, the indicator mechanism may include auditory feedback, such as clicking sounds, that are created when the user advances the perforation device 510 past certain points within the device 505. In certain embodiments, each indicator of the indicator mechanism may correspond to one millimeter of advancement by the perforation device 510. In certain embodiments, each indicator of the indicator mechanism may correspond to less than one millimeter of advancement by the perforation device 510. In certain embodiments, each indicator of the indicator mechanism may correspond to two millimeters of advancement by the perforation device 510. In other embodiments, any other application of force or motion may be translated into the lateral movement of the perforation device 510. In still other embodiments, the indicator mechanism may include at least one or more markings on the handle 524 representing the protrusion length of the perforation device 510. In an embodiment, as shown in FIGS. 5A-5C, as the first/top portion of the needle advancement mechanism 530 that is protruding from the handle 524 of the device 505 is longitudinally advanced by a user, the perforation device 510 is also longitudinally extended from the distal end portion 522 of the delivery device body 520.

In some embodiments, as best shown in FIG. 3B, having an electrocautery device and/or a needle advancement mechanism incorporated on the delivery device in which a perforation device is extended through the device lumen allows a single operator to be able to more easily perform many of the tasks required in an operation. In an embodiment, these tasks may include holding the dilator in place, holding a sheath, applying energy to the perforation device to be able to puncture a target site, advancing the perforation device towards the target site, perforating the target site, among others. In an embodiment, as shown, the user may advance the perforation device with their thumb while also simultaneously starting or stopping RF energy as needed. In an embodiment, all steps of the transseptal procedure from drop down of the assembly to puncturing of the target site can be achieved by a single operator without adjusting hand positions on the assembly.

In an embodiment, a device for perforating a tissue within a heart is disclosed. In some embodiments, the device includes a dilator comprising an elongated body having a body length, a proximal end and an opposite distal end portion terminating in a distal tip, a handle having a handle length and connected to the proximal end of the elongated body, and a dilator lumen extending through the handle and the elongated body and dimensioned to slidingly receive a perforation device having a perforation device body, a perforation device proximal end and an opposite perforation device distal end portion terminating in a perforation device distal tip. In an embodiment, the dilatory body length and the handle length together define a dilator length. In an embodiment, the device may also include an electrocautery device removably positioned within a void on the dilator handle and connected to an energy source, the electrocautery device having a conductive member positioned inside the dilator handle and in physical communication with the perforation device positioned within the dilator lumen. In an embodiment, when the electrocautery device is activated, energy is delivered from the electrocautery device through the conductive member and to the distal tip of the perforation device.

In an embodiment, a method for perforating a tissue within a heart includes providing a delivery device comprising an elongated body having a body length, a proximal end and an opposite distal end portion terminating in a distal tip, a handle having a handle length and connected to the proximal end of the elongated body, and a lumen extending through the handle and the elongated body and dimensioned to slidingly receive a perforation device having a perforation device body, a perforation device proximal end and an opposite perforation device distal end portion terminating in a perforation device distal tip. In an embodiment, the delivery device body length and the handle length together define a delivery device length. In further embodiments, the method for perforating a tissue within a heart also includes providing an electrocautery device removably positioned within a void on the handle and connected to an energy source, the electrocautery device having a conductive member positioned inside the handle and in physical communication with the perforation device positioned within the lumen. In some embodiments, when the electrocautery device is activated, energy is delivered from the electrocautery device through the conductive member and to the distal tip of the perforation device.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for perforating a tissue within a heart, the system comprising:
a delivery device comprising an elongated body having a body length, a proximal end and an opposite distal end portion terminating in a distal tip, a handle having a handle length and connected to the proximal end of the elongated body, and a lumen extending through the handle and the elongated body and dimensioned to slidingly receive a perforation device having a perforation device body, a perforation device proximal end and an opposite perforation device distal end portion terminating in a distal tip, wherein the delivery device body length and the handle length together define a delivery device length; and
an electrocautery device removably positioned into a void on the handle and connected to an energy source, the electrocautery device having a conductive member positioned inside the handle and in physical communication with the perforation device positioned within the lumen;
wherein when the electrocautery device is activated, energy is delivered from the electrocautery device through the conductive member and to the distal tip of the perforation device.

2. The system of claim 1, wherein the electrocautery device is an accessory device.

3. The system of claim 1, wherein the delivery device is a dilator.

4. The system of claim 1, wherein:
the perforation device includes at least one or more grooves located on the perforation device body; and
the conductive member aligns with one of the at least one or more grooves within the perforation device body to create a physical communication with the perforation device.

5. The system of claim 1, wherein when the electrocautery device is activated the physical communication between the conductive member and the perforation device allows for electrical communication with the distal end of the perforation device to facilitate radiofrequency puncture of a tissue within the heart.

6. The system of claim 1, further comprising a needle advancement mechanism positioned on the delivery device, and wherein the needle advancement mechanism is configured to interact with the perforation device and advance the perforation device longitudinally within the lumen.

7. The system of claim 1, wherein the electrocautery device includes a first button, and wherein the electrocautery device is activated when a user presses the first button.

8. The system of claim 1, wherein the electrocautery device includes a second button, and wherein when the second button is pressed by a user the electrocautery device stops delivery of energy to the conductive member.

9. The system of claim 1, wherein the perforation device is insulated along a portion of the perforation device body.

10. The system of claim 1, wherein the electrocautery device is held in place within the void of the handle by friction.

11. A device for perforating a tissue within a heart, the device comprising:
a dilator comprising an elongated body having a body length, a proximal end and an opposite distal end portion terminating in a distal tip, a handle having a handle length and connected to the proximal end of the elongated body, and a dilator lumen extending through the handle and the elongated body and dimensioned to slidingly receive a perforation needle having a perforation needle body, a perforation needle proximal end and an opposite perforation needle distal end portion terminating in a distal tip, wherein the dilatory body length and the handle length together define a dilator length;
an electrocautery device removably positioned into a void on the dilator handle and connected to an energy source, the electrocautery device having a conductive member positioned inside the dilator handle and in physical communication with the perforation needle positioned within the dilator lumen; and
a needle advancement mechanism positioned on the dilator and in physical communication with the perforation device, the needle advancement mechanism configured to advance the perforation needle longitudinally within the dilator lumen;
wherein when the electrocautery device is activated, energy is delivered from the electrocautery device through the conductive member and to the distal tip of the perforation device.

12. The device of claim 11, wherein the electrocautery device is an accessory device.

13. The device of claim 11, wherein:
the needle includes at least one or more grooves located on the needle body; and
the conductive member aligns with one of the at least one or more grooves within the needle body to create a physical communication with the perforation device.

14. The device of claim 11, wherein:
the needle includes at least one or more grooves located on the needle body;
the needle advancement mechanism is a knob having a first portion protruding from the dilator and a second portion positioned within the dilator lumen; and
when the knob aligns with one of the at least one or more grooves within the needle body, a user longitudinally advances the first portion of the knob across the dilator thereby advancing the needle longitudinally within the dilator lumen.

15. The device of claim 11, wherein the needle advancement mechanism is a dial having a traction element, and wherein friction applied by the dial to the needle advances the needle longitudinally within the dilator lumen.

16. The device of claim 11, wherein the needle advancement mechanism is an accessory device.

17. The device of claim 11, wherein the needle is insulated along a portion of the needle body.

18. The device of claim 11, wherein the electrocautery device is held in place within the void of the dilator handle by at least one or more actuatable elements which prevent unintended dislodgement of the electrocautery device.

19. The device of claim 18, wherein the at least one or more actuatable elements include latches, switches and screw features.

20. A method for perforating a tissue within a heart, the method comprising:
providing a delivery device comprising an elongated body having a body length, a proximal end and an opposite distal end portion terminating in a distal tip, a handle having a handle length and connected to the proximal end of the elongated body, and a lumen extending through the handle and the elongated body and dimensioned to slidingly receive a perforation device having a perforation device body, a perforation device proximal end and an opposite perforation device distal end portion terminating in a perforation device distal tip, wherein the delivery device body length and the handle length together define a delivery device length; and
providing an electrocautery device removably positioned into a void on the handle and connected to an energy source, the electrocautery device having a conductive member positioned inside the handle and in physical communication with the perforation device positioned within the lumen;
wherein when the electrocautery device is activated, energy is delivered from the electrocautery device through the conductive member and to the distal tip of the perforation device.
